(19) United States
(12) Patent Application Publication
(10) Pub. No.: US 2025/0046424 A1
(43) Pub. Date: Feb. 6, 2025

(54) DEVICE AND METHOD FOR PROVIDING CUSTOMIZED PET SERVICE BASED ON INTESTINAL MICROBIOME ANALYSIS

(71) Applicant: FRESHOUR INC., Chungju-si (KR)

(72) Inventor: Kyeongho IM, Chungju-si (KR)

(73) Assignee: FRESHOUR INC., Chungju-si (KR)

(21) Appl. No.: 18/793,379

(22) Filed: Aug. 2, 2024

(30) Foreign Application Priority Data
Aug. 4, 2023 (KR) 10-2023-0102060

(51) Int. Cl. G16H 20/60; C12Q 1/04; G16H 10/60

(52) U.S. Cl. CPC G16H 20/60; C12Q 1/04; G16H 10/60

(57) ABSTRACT

The present invention relates to a device and method for providing customized services for pets based on gut microbiome analysis.

### CROSS-REFERENCE TO RELATED APPLICATIONS

The present application claims the benefit of priority to Korean Patent Application No. 10-2023-0102060 filed on Aug. 4, 2023, the entire of which is hereby incorporated by reference.

### BACKGROUND OF THE INVENTION

In Korea, as well as globally, the number of people raising pet dogs is rapidly increasing. As pet dogs are recognized as family members, homemade food and vegetarian diets are sometimes provided, replacing commercial products available in the market. However, this poses a risk of exposing pet dogs to various diseases, necessitating a precise understanding and caution.

Diet is the most important factor influencing the composition of gut microorganisms, and changes in the composition of gut microorganisms are known to affect the production of metabolites. Since diet influences the composition and changes of gut microorganisms, an approach to diet based on a precise understanding of pet dogs is necessary.

Although there have been some studies on using gut microbiome to manufacture animal feed, research in the field of early diagnosis related to disease occurrence is still in its early stages. The conventional disease diagnosis techniques are mostly developed for diagnosing diseases in humans rather than pets and have the disadvantage of requiring invasive blood sampling for diagnosis.

In addition, due to differences between individuals and variations in the distribution of gut microorganisms based on the composition and type of feed, there is a need for the development of a customized analysis system that can meet individual nutritional deficiencies through microbial community analysis. However, conventional technology has not adequately satisfied this requirement.

Therefore, there is an urgent need for the development of a technology that can non-invasively collect samples for gut microbiome analysis from pets, identify the gut condition information (gut microbiome status) and existing diseases (ailments) of the pets based on the collected samples, and provide customized services such as tailored pet food and recipe recommendations for each individual pet based on the identified information.

### SUMMARY OF THE INVENTION

The present invention is to solve the problems of the prior art by providing a device and method for non-invasively collecting samples for gut microbiome analysis from pets, identifying the gut condition information (gut microbiome status) and existing diseases (ailments) of the pets based on the collected samples, and offering customized services such as customized pet food and recipe recommendations for each individual pet based on the identified information.

However, the technical objectives of the embodiments of the present invention are not limited to the technical objectives mentioned above, and other technical objectives may exist.

As one technical feature to achieve the above-mentioned objectives, in one aspect of the present invention, a device for providing customized services for pets based on gut microbiome analysis of the present invention may include an analysis unit for deriving gut condition information of the pet through gut microbiome analysis, and a service providing unit for providing customized services for the pet based on the gut condition information.

In addition, the analysis unit can collect a stool sample from the pet using a pre-manufactured diagnostic device, perform gut microbiome analysis based on the collected stool sample, and analyze information about microorganisms, including beneficial and harmful bacteria in the pet's gut, as the gut condition information.

In another aspect of the present invention, a device for providing customized services for pets based on gut microbiome analysis may further comprise a construction unit for establishing a microbiome-based community distribution map through the analysis of stool samples from multiple pets, wherein the analysis unit derives the gut condition information using the established community distribution map.

In addition, the service providing unit can provide customized recipe information or information about customized pet food products manufactured based on the customized recipe information, as the customized service according to the gut condition information.

In still another aspect, a device for providing customized services for pets based on gut microbiome analysis according to an embodiment of the present invention may further comprise an acquisition unit to obtain pet information, including at least one of the breeds, age, weight, eating habits, and activity level of the pet, from a user terminal. The service providing unit can provide the customized service by further considering the pet information.

In yet another aspect of the present invention, a device for providing customized services for pets based on gut microbiome analysis may further comprise a subscription management unit to regularly deliver customized pet food products on user-specified delivery dates when a subscription request for information about customized pet food products is made from a user terminal.

Meanwhile, in one aspect of the present invention, a method for providing customized services for pets based on gut microbiome analysis using the device for providing customized services for pets based on gut microbiome analysis comprises: deriving gut condition information of the pet through gut microbiome analysis in the analysis unit; and providing a customized service for the pet based on the gut condition information in the service providing unit.

The technical features for solving the above-mentioned problems are merely exemplary and should not be construed as limiting the present invention. In addition to the above-mentioned exemplary embodiments, additional embodiments may exist in the drawings and the detailed description of the invention.

According to the technical features of the present invention described above, by providing a device and method for customized services for pets based on gut microbiome analysis, it is possible to non-invasively collect samples for gut microbiome analysis from pets using a pre-manufactured diagnostic device. Based on the collected samples, the gut condition information (gut microbiome status) and existing diseases (ailments) of the pets can be identified, and customized services such as customized pet food and recipe recommendations can be provided to individual pets based on the identified information.

However, the effects obtainable from the present invention are not limited to the effects mentioned above, and other effects may also exist.

### BRIEF DESCRIPTION OF THE DRAWINGS

FIG. 1 is a diagram illustrating the schematic configuration of a system for providing customized services for pets based on gut microbiome analysis according to one embodiment of the present invention.

FIG. 2 is a diagram explaining the process of constructing a community distribution map by the construction unit within the device for providing customized services for pets based on gut microbiome analysis according to one embodiment of the present invention

FIG. 3 is a diagram schematically illustrating the product shape of a diagnostic device included in the system for providing customized services for pets based on gut microbiome analysis according to one embodiment of the present invention.

FIG. 4 is a diagram explaining the process of producing and providing probiotics as a customized pet food product through gut microbiome analysis of pets by the device for providing customized services for pets based on gut microbiome analysis according to one embodiment of the present invention.

FIGS. 5A-5D are diagrams illustrating examples of visualizing data for each sample by the device for providing customized services for pets based on gut microbiome analysis according to one embodiment of the present invention.

FIG. 6 is a diagram explaining the customized services provided by the device for providing customized services for pets based on gut microbiome analysis according to one embodiment of the present invention.

FIG. 7 is a flowchart illustrating the operation of a method for providing customized services for pets based on gut microbiome analysis according to one embodiment of the present invention.

### DETAILED DESCRIPTION OF THE EMBODIMENTS

The following detailed description of the embodiments of the present invention is provided to enable those skilled in the art to practice the invention easily, with reference to the accompanying drawings. However, the present invention may be embodied in various forms and is not limited to the embodiments described herein. In the drawings, parts unrelated to the description have been omitted for clarity, and similar reference numerals are used for similar parts throughout the specification.

Throughout the specification of the present invention, when a part is described as being ‘connected’ to another part, this includes not only cases where they are ‘directly connected’ but also cases where they are ‘electrically connected’ or ‘indirectly connected’ with another element interposed therebetween.

Throughout the specification of the present invention, when a material is described as being ‘on,’ ‘above,’ ‘at the top of,’ ‘under,’ ‘below,’ or ‘at the bottom of’ another material, this includes not only cases where one material is in contact with another material but also cases where another material is present between the two materials.

Throughout the specification of the present invention, when a part is described as ‘including’ a certain component, this means that it can further include other components, unless specifically stated otherwise, and does not exclude other components.

Throughout the specification of the present invention, some of the operations or functions described as being performed by a terminal, device, or apparatus may be performed by a server connected to the terminal, device, or apparatus. Similarly, some of the operations or functions described as being performed by a server may be performed by a terminal, device, or apparatus connected to the server.

Throughout the specification of the present invention, the term ‘at least one’ can be defined as a term that includes both singular and plural forms. Even if the term ‘at least one’ is not present, it is evident that each component may exist in singular or plural form and can mean either singular or plural. Additionally, whether each component is provided in singular or plural form can vary depending on the embodiment.

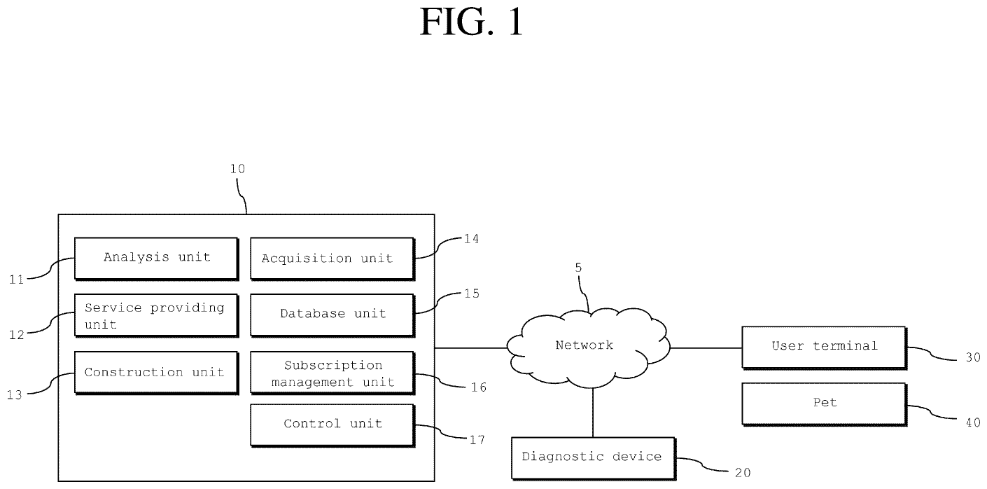

FIG. 1 is a diagram illustrating the schematic configuration of a system for providing customized services for pets based on gut microbiome analysis (100) according to one embodiment of the present invention.

For the convenience of explanation, the system for providing customized services for pets based on gut microbiome analysis (100) according to one embodiment of the present invention will be referred to as ‘the system (100).’ Additionally, the system (100) may include a device for providing customized services for pets based on gut microbiome analysis (10) according to one embodiment of the present invention, which, for the convenience of explanation, will be referred to as ‘the device (10)’.

Referring to FIG. 1, the system (100) may include the device (10), a diagnostic device (20), a user terminal (30), and a pet (40).

The device (10) is a device for providing customized services for pets based on gut microbiome analysis. It can be a device or server that provides web pages, app pages, programs, or applications related to providing customized services for pets based on gut microbiome analysis. In this context, the programs, applications, services, and platforms related to providing customized services for pets based on gut microbiome analysis, provided by the device (10), will be referred to as ‘the program,’ ‘the app,’ ‘the service,’ and ‘the platform,’ respectively, for convenience of explanation. The device (10) can provide the program or the app to a user terminal (30), allowing the user to access the service (customized services for pets based on gut microbiome analysis).

The diagnostic device (20) is designed to collect stool samples from the pet (40) and can be a diagnostic device that identifies (derives) the gut condition information (gut microbiome status) and existing diseases (ailments) of the pet (40) using the collected stool samples. The diagnostic device (20) can be a portable diagnostic device. Such a diagnostic device (20) may also be referred to by terms such as a microbiome platform-based diagnostic device for pet diseases, a diagnostic device for gastrointestinal diseases, a health diagnostic device for pets, a stool sample-based diagnostic device, a stool sample-based microbiome analysis diagnostic device, a stool sample-based gut microbiome diagnostic device, or a stool sample-based NGS (Next Generation Sequencing) diagnostic device.

In one embodiment, the device (10) may provide the diagnostic device (20) to users utilizing the service, either for a fee or free of charge, allowing users to receive customized services for their pets (40). In one embodiment, users can purchase the diagnostic device from the device (10) by making a payment for the diagnostic device's predetermined sale price set by the administrator of the device (10) using the user terminal (30).

The user terminal (30) refers to a terminal owned by the user who uses the device (10) (using the service). The device (10) may allow the user to use the service (customized services for pets based on gut microbiome analysis) by accessing the program or app through the user terminal (30) with or without registering as a member. The user can be any person regardless of age or gender.

The user terminal (30) may include, for example, all types of wired and wireless communication devices such as a PCS (Personal Communication System), GSM (Global System for Mobile communication), PDC (Personal Digital Cellular), PHS (Personal Handy-phone System), PDA (Personal Digital Assistant), IMT (International Mobile Telecommunication)-2000, CDMA (Code Division Multiple Access)-2000, W-CDMA (Wideband Code Division Multiple Access), Wibro (Wireless Broadband Internet) terminal, smartphone, smart pad, tablet PC, notebook, wearable device, desktop PC, and the like, but is not limited thereto.

The pet (40) may be, for example, a companion animal raised by the user. The pet (40) may include dogs, cats, hedgehogs, and the like. However, it is not limited to the aforementioned types, and various kinds of animals can be considered as pets (40) in the system (100). In the present invention, for example, the pet (40) is a user's pet, and a puppy/a dog (companion dog) is used as an example.

The device (10) can send and receive data by being connected, respectively, to the user terminal (30) and the diagnostic device (20) through a network (5). Additionally, the control unit (17) within the device (10) can control the operations of the user terminal (30) and the diagnostic device (20) as well as the operations of each part within the device (10).

The network (5) may include, for example, a 3GPP (3rd Generation Partnership Project) network, LTE (Long Term Evolution) network, WIMAX (World Interoperability for Microwave Access) network, Internet, LAN (Local Area Network), Wireless LAN (Wireless Local Area Network), WAN (Wide Area Network), PAN (Personal Area Network), Bluetooth network, NFC (Near Field Communication) network, satellite broadcasting network, analog broadcasting network, DMB (Digital Multimedia Broadcasting) network, and the like, but is not limited thereto, and may include various wired/wireless communication networks.

In the example shown in FIG. 1, each component within the system (100) (for example, the user terminal (30), the diagnostic device (20), and the pet (40)) is illustrated as being provided (included) individually. However, this is merely an example to aid in understanding the present invention, and the present invention is not limited to this. In another example, the system (100) may include multiple instances of each of the aforementioned components, and the descriptions provided for each of the aforementioned components can be applied similarly to the cases where multiple instances of each component (i.e., multiple user terminals, multiple diagnostic devices, multiple pets) are provided. The following is a more detailed description of the device (10).

The device (10) may include an analysis unit (11), a service providing unit (12), a construction unit (13), an acquisition unit (14), a database unit (15), a subscription management unit (16), and a control unit (17).

The analysis unit (11) can derive gut condition information of the pet (40) through gut microbiome analysis. The term “microbiome” collectively refers to various microorganisms such as bacteria and viruses living in living creature's bodies, which change over time and affect human health. In this invention, the term “microbiome” may be used interchangeably with “microorganisms.” Accordingly, the analysis unit (11) can derive the gut condition information of the pet (40) by performing gut microbiome (microorganism) analysis on the pet (40).

The analysis unit (11) can collect stool samples from the pet (40) using a pre-manufactured diagnostic device (20), perform gut microbiome analysis on the collected stool samples, and derive analysis result information (microorganism analysis result information) about microorganisms, including beneficial and harmful bacteria in the pet's (40) gut, as the gut condition information of the pet (40). Here, the diagnostic device (20) may be a diagnostic device manufactured (produced) and provided by a manufacturing unit (not shown) within the device (10), and may be referred to by terms such as a stool-based gut microorganism diagnostic kit. The pre-manufactured diagnostic device (20) can be a portable diagnostic device designed to be of portable size.

The gut condition information derived by the analysis unit (11) refers to the current gut microorganism (microbiome) status information of the pet (40), which can be understood as the gut microbiome analysis result information (gut microorganism analysis result information) derived through the gut microbiome analysis. This gut condition information may include at least one of the type, distribution, community composition, and ratio of gut microorganisms. Here, information on distribution, community composition, and ratio may refer to information according to the types of gut microorganisms (i.e., information on the distribution, community composition, and ratio of each type of microorganism) or information according to beneficial and harmful bacteria (i.e., information on the distribution, community composition, and ratio of beneficial and harmful bacteria).

In addition, the analysis unit (11) can derive not only the current gut condition information of the pet (40) but also the current disease information of the pet (40) through gut microbiome analysis of the collected stool samples of the pet (40). Here, current disease information refers to information about diseases (ailments) currently possessed by the pet (40), particularly the types of the diseases. This current disease information may also be referred to by terms such as current disease type information.

The construction unit (13) can establish (generate) a microbiome-based community distribution map through the analysis of stool samples from multiple pets. The analysis unit (11) can derive the gut condition information of the pet (40) using the community distribution map established by the construction unit (13). Additionally, the analysis unit (11) can derive the gut condition information and current disease information of the pet (40) using the established community distribution map.

The community distribution map established by the construction unit (13) may refer to mapped information on the community distribution of microbiomes (microorganisms) based on the analysis of stool samples from multiple pets and categorized by species, age group, weight, and diseases (ailments).

Specifically, to establish (generate) the community distribution map, the construction unit (13) collects stool samples from multiple pets, categorizes the collected stool samples into a first group, including stool samples from normal pets, and a second group, including stool samples from diseased pets, and performs gut microbiome analysis on each of these groups. This process performed by the construction unit derives gut microorganism analysis result information for both the normal pets and the diseased pets, which is then used to establish (generate) the community distribution map.

When collecting stool samples from multiple pets, the construction unit (13) can collect the stool samples categorized by species, age group, and weight of the pets. In the above description, normal pets refer to pets that do not possess any diseases (ailments), where diseases may be referred to by terms such as illnesses, metabolic diseases, gastrointestinal metabolic diseases, etc. Diseased pets refer to pets that possess at least one type of disease (ailment).

The community distribution map established by the construction unit (13) can include a first map of the microbial community distribution for normal pets and a second map of the microbial community distribution for diseased pets. The first map may include pie chart information of microbiome analysis categorized by species, age, and weight for healthy breeds (i.e., normal pets), and the second map may include pie chart information of microbiome analysis categorized by species, age, and weight for breeds with metabolic diseases (i.e., diseased pets).

A pie chart, also known as a circle chart or circle graph, is represented by sectors (fan-shape) based on the proportion of different categories within a whole circle, and the central angle of each sector is proportional to its proportion.

The analysis unit (11), for example, can derive gut condition information (i.e., microorganism analysis result information) of the pet (40) by performing gut microbiome analysis on the pet (40) using a pre-manufactured diagnostic device (20). By comparing the derived gut condition information of the pet (40) with the community distribution map established by the construction unit (13), the analysis unit (11) can derive the current disease information of the pet (40) based on the established community distribution map. Specifically, the current disease information may include information on whether the pet (40) currently has a disease and, if so, the type of disease possessed.

Subsequently, the service providing unit (12) can provide a customized service for the pet (40) based on the gut condition information derived by the analysis unit (11). Specifically, the service providing unit (12) can provide a customized service for the pet (40) based on the gut condition information and the current disease information of the pet (40) derived by the analysis unit (11) to the user terminal (30). This allows the user to receive and utilize a customized service tailored to the pet (40) from the service providing unit (12).

The service providing unit (12) can provide customized recipe information or information about customized pet food products manufactured based on the customized recipe information as a customized service to the user (via the user terminal (30)) based on the gut condition information of the pet (40) derived by the analysis unit (11). Specifically, the service providing unit (12) can provide customized recipe information or information about customized pet food products manufactured based on the customized recipe information as a customized service to the user terminal (30) based on the current status information of the pet (40), including the gut condition information and current disease information derived by the analysis unit (11).

In this case, the customized pet food products can be, for example, a pet food product (i.e., food for pets) manufactured by the pet food manufacturing unit (not shown) within the device (10). The pet food manufacturing unit (not shown) can manufacture (create, produce) a pet food product corresponding to the customized recipe information based on the customized recipe information provided by the service providing unit (12). Here, the customized pet food product can include fresh food (raw and cooked food) manufactured based on fresh ingredients (i.e., fresh ingredients that correspond to at least one of domestic, organic, and eco-friendly). In other words, the customized pet food product can include feed (fresh feed), but it is not limited to this, and various types of pet products, such as feed, snacks, and nutritional supplements, can be included as types of customized pet food products.

The user terminal (30) can receive customized services from the service providing unit (12). If the customized service provided by the service providing unit (12) is customized recipe information, the user can directly prepare food containing the ingredients and nutrients included in the customized recipe information based on the customized recipe information and then provide it as customized pet food for the pet (40) to eat.

Alternatively, if the customized service provided by the service providing unit (12) is information about customized pet food products, the user can check the provided information about the customized pet food products and, if desired, select and purchase the customized pet food products. In this case, for example, if the control unit (17) within the device (10) detects that the payment for the information about the customized pet food products has been completed on the user terminal (30), the control unit (17) can have the customized pet food products manufactured by the pet food manufacturing unit (not shown) within the device (10). The customized pet food products manufactured by the pet food manufacturing unit (not shown) can then be delivered to the user, who can subsequently provide the delivered customized pet food products to the pet (40) to eat.

The acquisition unit (14) can obtain pet information, including at least one of the breeds, age, weight, eating habits, and activity level of the pet (40), from the user terminal (30). In other words, the pet information may include at least one of breed information, age information, weight information, eating habits information, and activity level information. Here, activity level information can be information related to the activity level of the pet (40) obtained through a wearable device that can be worn on a part of the pet's (40) body, such as the number of steps taken by the pet (40). Additionally, pet information may also include information on the presence of diseases, the type of diseases if present, and allergy information, in addition to the aforementioned information (breed, age, weight, eating habits, activity level).

The acquisition unit (14) can store the pet information obtained from the user terminal (30) in the database unit (15). The database unit (15) can store not only the pet information obtained from the user terminal (30) but also various information considered by the device (10).

The service providing unit (12) can provide customized services to the user (via the user terminal (30)) by considering not only the gut condition information and current disease information mentioned above but also the pet information obtained by the acquisition unit (14).

The subscription management unit (16) can regularly deliver customized pet food products (i.e., customized pet food products corresponding to the information about customized pet food products for which a subscription request has been made by the user) to the user on pre-assigned delivery dates if a subscription request for information about customized pet food products is made from the user terminal (30).

Here, the information about customized pet food products refers to the information about customized pet food products provided as a customized service by the service providing unit (12). For example, when performing a subscription request for information about customized pet food products, the user can enter subscription request information, which may include at least one of desired delivery dates (delivery dates), desired delivery frequency, desired delivery address, and regular payment methods.

Accordingly, when the regular subscription request, including the subscription request information, is made related to the customized pet food products from the user terminal (30), the subscription management unit (16) can perform automatic payments with a regular payment method based on the subscription request information, such as the desired delivery date (pre-assigned by the user) for the desired delivery frequency, and arrange for the customized pet food products to be delivered to the desired delivery address. For example, the desired delivery date can be set to any date from the 1st to the 31st of the month, such as the 1st, 5th, 10th, etc., and the desired delivery frequency can be set to 2 weeks, 1 month, 2 months, etc.

The control unit (17) can control the operations of each part within the device (10). Additionally, the control unit (17) can control the operations of the user terminal (30) and the diagnostic device (20) that are connected to the device (10) through the network (5).

As described above, the device (10) can analyze the data of gut microbiome (gut microorganisms) based on the species, age, and weight of the pet (40), construct platform (database) the community distribution information of beneficial and harmful bacteria and establish (platformization) the community distribution map by further analyzing the correlation with diseases based on the established information. This allows for the manufacture of individually customized functional pet food products (e.g., feed) tailored to each pet (40) on a 1:1 basis, which can then be provided as a customized service to the user. Through this, the user can receive a more advanced pet-specific healthcare service compared to conventional technologies.

In this case, the construction unit (13) can establish (generate) a microbiome-based community distribution map through the analysis of stool samples from multiple pets, as previously described. The detailed description of this is as follows.

For providing the customized service, the construction unit (13) can perform the following steps when establishing the community distribution map: i) establishing the analysis basis by securing gut microbiome samples from multiple pets categorized by species, age group, and weight in the first step; ii) analyzing the community distribution of beneficial gut microorganisms using Next Generation Sequencing (NGS) in the second step; iii) analyzing the distribution of gut microorganisms related to diseases in the third step; and iv) organizing the data and platformizing the microbial map (i.e., community distribution map) in the fourth step.

### <In the First Step>

In the first step, the construction unit (13) can collect stool samples from multiple pets categorized by species, age group, and weight through enterprises and classify the collected stool samples into healthy pet stool samples and diseased pet stool samples (those with metabolic diseases), thereby securing gut microbiome samples from both healthy and diseased pets and establishing conditions for the analysis samples. In this step, the construction unit (13) can prepare stool samples separately for normal pets and diseased pets with more than two types of metabolic diseases (e.g., constipation and diarrhea). That is, in the first step, the construction unit (13) can classify the collected stool samples into a first group and a second group, where the first group includes stool samples from normal pets (those without diseases), and the second group includes stool samples from diseased pets (those with at least two types of diseases).

### <In the Second Step>

In the second step, the construction unit (13) can analyze the community distribution of beneficial gut microorganisms using Next Generation Sequencing (NGS). This involves performing NGS analysis on the collected stool samples to derive NGS analysis results and analyzing and listing the distribution of beneficial gut microorganisms from the derived NGS analysis results.

Specifically, in the second step, the construction unit (13) can perform NGS analysis on the stool samples of the first group to derive the NGS analysis results of normal pets and analyze and list the distribution (i.e., the first distribution) of beneficial gut microorganisms in normal pets from the derived NGS analysis results. Additionally, the construction unit (13) can perform NGS analysis on the stool samples of the second group to derive the NGS analysis results of diseased pets and analyze and list the distribution (i.e., the second distribution) of beneficial gut microorganisms in diseased pets from the derived NGS analysis results.

Here, beneficial gut microorganisms may include, for example, Lactobacillus, Firmicutes, Enterococcus faecium, Clostridium, Proteobacteria, to etc. Lactobacillus refers beneficial bacteria that help the gut environment, Firmicutes are microorganisms related to dietary habits, Enterococcus faecium helps maintain the gut environment but can develop antibiotic resistance if excessive, Clostridium is related to gut diseases, and Proteobacteria is related to gut inflammation.

### <In the Third Step>

In the third step, the construction unit (13) can analyze the distribution of gut microorganisms related to diseases by comparing the first and second distributions (i.e., the distribution of beneficial gut microorganisms in normal pets and diseased pets) derived in the second step.

### <In the Fourth Step>

In the fourth step, the construction unit (13) can organize the data and platformize the microbial map (i.e., community distribution map). Specifically, i) the construction unit (13) can create (generate) a pie chart of microbiome analysis by species, age, and weight for normal pets as ‘the first map of microbial community distribution for normal pets,’ and ii) create (generate) a pie chart of microbiome analysis by species, age, and weight for diseased pets as ‘the second map of microbial community distribution for diseased pets.’ By creating (generating) the first and second maps in this way, the construction unit (13) can establish (generate) a community distribution map that includes the first and second maps.

Additionally, the construction unit (13) can compare and analyze the first map created for the first group and the second map created for the second group (i.e., compare and analyze the microbiome analysis results for healthy and diseased pets) to list the microbial communities that differ between the first and second maps (i.e., the differential microorganisms).

The analysis unit (11) can derive, for example, the current disease information of the pet (40) by comparing (contrasting) the gut condition information of the pet (40) (i.e., the gut microbiome analysis information of the pet (40)) derived using the diagnostic device (20) with the community distribution map established by the construction unit (13). Subsequently, the service providing unit (12) can provide customized services to the user based on the current disease information and gut condition information derived by the analysis unit (11).

The present invention (i.e., the system (100) and the device (10)) can manufacture (produce, develop) and provide a diagnostic device (20) capable of microbiome-based gut microbiome analysis of pets (40) through NGS analysis of pet stool samples. Using this diagnostic device (20), the gut condition information and current disease information of the pet (40) can be derived based on gut microbiome analysis anytime and anywhere, and customized services can be provided based on this information.

In general, conventional research focuses on analyzing the human gut microbiome and forming microbial communities. In contrast, the present invention (the system (100) and the device (10)) can derive the metabolic and immune diseases (current disease information) of pets (40) by analyzing the five major beneficial bacteria and two types of harmful bacteria through stool-based gut microbiome analysis of pets (40).

The present invention can provide technology for improving the health of pets throughout their lifecycle based on disease-specific research for collecting microbial community data and can provide pet-specific solutions (customized services) to users based on the analysis of the gut microbial community characteristics and disease correlations by species and age, etc., of the pets.

Through the provision of the service, the present invention (i.e., the system (100) and the device (10)) can improve the quality of mental life for pet owners by promoting the health and longevity of pets through the construction of a microbial ecosystem map. Additionally, the present invention allows for the preemptive acquisition of technology in pet healthcare and provides individually customized functional and prescription pet food based on the basic gut microbiome information of different breeds, allowing for the intake of nutrients lacking by breed and age group. Furthermore, the present invention provides a diagnostic device (20) as a biomarker and disease diagnostic device linked to diseases for the development of pet health diagnostic devices. The present invention can generate gut microbiome distribution data for different breeds and ages of normal pets (i.e., the first map) and gut microbiome distribution data for pets with metabolic diseases (i.e., the second map), and through the comparison of the first map and the second map, generate comparative data on the gut microbiome distribution between normal and diseased pets (i.e., information listing the differential microbial communities).

### <The Relationship Between Diet and Gut Microorganisms in Pets>

Meanwhile, the relationship between diet and gut microorganisms in pets is as follows.

In Korea and globally, the population of people raising pet dogs is rapidly increasing. As pet dogs are recognized as family members, homemade food and vegetarian diets are sometimes provided, replacing commercial products available in the market. However, this poses a risk of exposing pet dogs to various diseases, necessitating a precise understanding and caution.

Dogs lack amylase in their saliva and have a short gastrointestinal tract, characteristic of carnivores. However, through domestication, three genes (AMY2B, MGAM, SGLT1) have enabled dogs to digest carbohydrates and absorb glucose. Based on this, some argue that dogs, which diverged from the gray wolf (Canis lupus) 15,000 years ago and entered human living areas, should now be classified as omnivores. Previous studies have shown that diets containing carbohydrates increase the pH of stool samples and decrease ammonia, whereas dogs fed a high-protein diet based on by-products of pig fat production exhibited increased pH and persistent diarrhea symptoms.

Moreover, high-protein diets have been shown to decrease volatile fatty acids (VFAs) due to the decrease of propionic acid and acetic acid as intestinal metabolites, while increasing branched-chain fatty acids. Notably, studies have reported that dogs on high-protein diets show increased levels of calprotectin, a marker of inflammatory bowel disease.

Many animals, including dogs, have a vast microbial population of over 1,000 species in their gastrointestinal tract (GIT). Gut microorganisms consist of bacteria, archaea, and viruses present in the GIT and typically have a symbiotic relationship with the host.

Gut microorganisms are involved in metabolic activities, protection from pathogenic bacteria, and influencing the immune system, thereby playing a role in basic bodily functions and directly/indirectly in physiological functions.

In particular, the biogeographic characteristics of gut microorganisms vary with diet, and the density of microbial communities increases along the GIT. When analyzing the bacterial phyla in dog fecal samples using 16S rRNA analysis, the dominant groups in most dogs are Firmicutes, Proteobacteria, Fusobacteria, Bacteroidetes, and Actinobacteria. However, unique and stable yet distinct communities can also be identified in individual dogs.

Diet is the most crucial factor influencing the composition of gut microorganisms. Changes in the composition of gut microorganisms are known to affect the production of metabolites. Additionally, gut metabolites are significant factors that can influence the brain through systemic immune regulation and retrograde transport. Many related studies based on gut-brain bidirectional communication have been reported.

In Korea, the diet of pet dogs has evolved from providing leftover food to nutritionally balanced commercial food such as kibble. Studies on the gut microbiome composition of natural diets and commercial diets have shown that a group of dogs fed a natural diet composed of approximately 90% meat and 10% vegetables had significantly higher numbers, richness, and diversity of gut microbial communities compared to those fed commercial kibble.

Many animals a vast community of gut form microorganisms that are ten times greater than their own genes, but these have evolved through different lifestyles and can be broadly classified as omnivorous, carnivorous, and herbivorous. This provides fundamental information that diet influences the composition and changes of gut microorganisms, and thus, a dietary approach based on a precise understanding of pets is necessary. Accordingly, the present invention aims to provide a customized service (e.g., providing customized pet food products) tailored to the current gut condition information and current disease information of the user's pet by analyzing the gut microbiome (microorganisms) of pet stool samples.

Specifically, for providing the customized service, the analysis unit (11) within the device (10) can derive the gut condition information of the pet (40). Then, by comparing the derived gut condition information with the community distribution map established by the construction unit (13), the analysis unit (11) can derive the current disease information, including the presence or absence of diseases and the types of diseases present in the pet (40). Subsequently, the service providing unit (12) can provide a customized service based on the current gut condition information and current disease information derived by the analysis unit (11). This involves, for example, i) identifying the microbiome community distribution information (i.e., information on the beneficial and harmful bacteria community distribution of the matching pet, including the types, quantities, and proportions of each beneficial and harmful bacteria present in the matching pet) of a matching pet based on the current condition of the pet (40) (which includes the breed, age, and weight information of the pet (40) pre-entered from the user terminal (30) and the current disease information derived by the analysis unit (11)) and the community distribution map established by the construction unit (13), ii) deriving information of the types and quantities of beneficial bacteria that are deficient or excessive in the current pet (40), as well as the types and quantities of harmful bacteria present in the current pet (40) by comparing the identified microbiome community distribution information of the matching pet with the gut condition information of the pet (40) (i.e., the current microbiome analysis result information, including the types and numbers of beneficial and harmful bacteria, and their proportions in the gut of the pet (40)), and iii) generating customized recipe information that includes nutrient information to increase (fulfill) the types and numbers of beneficial bacteria currently lacking in the pet (40) (especially to match the types and numbers of beneficial bacteria included in the microbiome community distribution information of the matching pet), decrease the types and numbers of currently excessive (overabundant) beneficial bacteria, or reduce the harmful bacteria present in the pet (40), and providing the customized recipe information to the user terminal (30) or providing information about customized pet food products manufactured based on the customized recipe information to the user terminal (30).

Meanwhile, the diversity gut of microorganisms decreases with age, which is known to result in weakened bodily functions, cognitive decline, and loss of physiological functions. In particular, changes in gut microorganisms observed in aging are associated with immunosenescence and chronic inflammation. Many diseases affect or are affected by gut microorganisms, leading to an imbalance of gut microorganisms, which in turn influences the progression, symptoms, and acceleration of diseases.

Changes in metabolites due to an imbalance of gut microorganisms affect innate immunity and immune regulation and can simultaneously influence diarrhea. Previous studies have analyzed the gut microorganisms of healthy dogs and those with non-hemorrhagic diarrhea (NHD), acute hemorrhagic diarrhea (AHD), or inflammatory bowel disease (IBD), identifying changes in Firmicutes, Proteobacteria, and Actinobacteria.

Changes in gut microorganisms also affect the production of short-chain fatty acids (SCFAs). Particularly, a decrease in Erysipelotrichaceae, Ruminococcus, and Blautia may reduce the production of SCFAs involved in immune function regulation. Additionally, a decrease in Faecalibacterium and unclassified Ruminococcaceae and an increase in C. perfringens in acute diarrhea may affect the reduction of propionic acid and the increase of butyric acid.

The pet care market in Korea is characterized by the development of the industrial ecosystem led by individuals and small and medium-sized enterprises (SMEs), including startups. Analysis of patent applicants in the related field shows that large enterprises account for only 2%, while individuals and SMEs account for 88%.

The pet care market can be broadly divided into three areas: 1) Pet Humanization, which refers to becoming pets as a member of a family; 2) Pet-Tech, which involves the integration of advanced technologies such as IoT and AI; and 3) Innovative Veterinary diagnostics and medical care providing rapid diagnosis and treatment. Among these, the health and healthcare sector for pets is gaining attention, with an increase in development tasks related to functional foods for pets and functional feed development as new growth industries.

Furthermore, wearable devices that allow easy pre-diagnosis of disease risks using digital technology and big data are being developed. Recent research efforts are aimed at manufacturing functional feed with a new formulation through the analysis of gut microbiome components in pets. However, most of these efforts have focused on feeds containing multiple nutrients and natural ingredients.

Additionally, while research is ongoing to manufacture safer feeds by enhancing the health and immunity of pets through individually tailored feeds based on the gut microbiome of each pet, research in the early diagnosis field related to disease occurrence is still in its initial stages.

However, most research has been developed based on technologies for diagnosing human diseases, and it has the disadvantage of requiring invasive blood sampling for diagnosis. Additionally, there are differences in the distribution of gut microorganisms due to individual differences and the combination and type of feed, necessitating the development of a customized analysis system to meet individual nutritional deficiencies based on microbial community analysis.

Therefore, the present invention (i.e., the system (100) and the device (10)) provides a technology that can identify the health status of pets (i.e., gut condition information and current disease information) through non-invasive gut microbiome analysis using pet stool samples. The technology involves constructing a microbiome platform analysis map based on the distribution and community analysis of gut microorganisms necessary for the individual customized pet food manufacturing and providing the technology to identify the pet's health status with non-invasive method.

The device (10) can manufacture and provide a diagnostic device (20) as a product for diagnosing pet gut microorganisms. Specifically, it can manufacture and provide the diagnostic device (20) as a stool-based diagnostic device and a biomarker through NGS analysis. The device (10) can perform platformization the types, distribution, and community of gut microorganisms through data-based pet gut microorganism diagnosis and provide a disease-linked customized dietary solution (customized service) based on the constructed community.

The device (10) can apply user convenience requirements to the product design and design safety products when manufacturing the diagnostic device (20), a diagnostic kit for pet gut microorganisms. It can also develop and apply biomarkers for detecting five major microorganisms.

To perform platformization the gut microbial community (i.e., to construct a community distribution map in the construction unit (13)), for example, the device (10) can generate pie charts of beneficial bacteria ratios in stool samples from breeds, age groups, and weights (normal pets), generate pie charts of harmful bacteria ratios in stool samples from breeds, age groups, and weights (normal pets), perform NGS analysis on stool samples from breeds with digestive and metabolic diseases (diseased pets), and then perform platformization a community distribution map based on the gut microbiome database of healthy breeds and age groups (i.e., generate the first map) and a community distribution map of microbiome distribution ratios in diseased breeds (i.e., generate the second map). As a result, it can construct a community distribution map that includes the first map and the second map.

Additionally, related to providing disease-linked customized dietary solutions (customized services), for example, the device (10) can provide a customized dietary solution as a customized service through NGS analysis of the pet's (40) stool. In addition, the device (10) can generate and provide a customized prescription diet (e.g., customized recipe information) linked to the current disease if the pet (40) has a disease (detected as having current disease information).

By providing a diagnostic device (20) capable of stool-based gut microorganism diagnosis, the device (10) can manufacture (develop) and provide the diagnostic device (20) as a stool-based gut microorganism diagnostic kit linked to the development of customized solutions (diets). This is significant given that most processes only proceed to analysis, are not user-friendly, and lack targeted customized solutions for each pet, thereby achieving significant progress in the pet healthcare industry and enhancing competitiveness.

Moreover, by performing platformization of pet gut microbial communities and microbiome information (i.e., constructing community distribution maps), the device (10) can express the distribution ratios of over five types of beneficial bacteria, the distribution ratios of at least two types of harmful bacteria, and the beneficial bacteria analysis ratios in samples from at least two breeds with digestive and metabolic diseases in pie charts. Considering that this has not been attempted domestically or internationally, the present invention can provide a precision diagnostic information system for pets, significantly contributing to the lifespan and health of pets and expecting synergies with related veterinary hospitals.

When measuring the ratio of beneficial bacteria through stool-based NGS analysis on samples from multiple pets, for example, the number of samples used may be 20 pets (unit) for the beneficial bacteria ratio pie chart, 20 pets (unit) for the harmful bacteria ratio pie chart, 10 pets (unit) for the analysis of beneficial bacteria in at least two types of digestive metabolic diseases, and 30 pets (unit) for constructing the gut microbiome analysis map.

The construction unit (13) can generate beneficial and harmful bacteria ratio pie charts for normal pets and harmful bacteria ratio pie charts for diseased pets through NGS analysis when constructing the community distribution map. Additionally, for example, by analyzing the community distribution ratio of at least five types of beneficial bacteria, the construction unit (13) can establish (generate, create) the community distribution map as a gut microbiome map.

Specifically, the construction unit (13) can, i) generate a beneficial bacteria ratio pie chart corresponding to the distribution ratio of more than five types of beneficial bacteria in stool samples from breeds categorized by species, age group, and weight through NGS analysis, using 20 samples, ii) generate a harmful bacteria ratio pie chart corresponding to the distribution ratio of at least two types of harmful bacteria in stool samples from breeds categorized by age group and weight through NGS analysis, using 20 samples, iii) generate a harmful bacteria ratio (diseased group) pie chart corresponding to the beneficial bacteria analysis ratio in stool samples from breeds with digestive metabolic diseases such as diarrhea and constipation, etc., using 10 samples, and iv) establish (generate, create) a community distribution map corresponding to the gut microbiome analysis map through the platformization of a community distribution map based on the gut microbiome database of healthy breeds and ages and the analysis of microbiome community distribution ratios in diseased breeds, using 30 samples.

Through the construction of the community distribution map, the device (10) can perform platformiztion the gut microbiome database of normal and diseased pets, and further manufacture and provide functional prescription pet food containing supplements specific to each disease as customized pet food products for pets (40) based on the differences between healthy and diseased pets.

The device (10) can perform platformization of pet gut microbiome data through the construction of the community distribution map and manufacture and provide customized functional and prescription pet food based on the platfromized basic gut microbiome information of breeds (e.g., the first map) to allow the intake of nutrients lacking by breed and age group. This enables the provision of precisely customized pet food products, supported by numerical evidence, rather than vaguely customized products, to users.

Furthermore, the device (10) can manufacture (create) and provide customized pet food products (e.g., feed) as functional prescription diets based on gut microorganism diagnosis. Unlike most commercial pet feeds that are mass-produced based on basic manufacturing methods, the customized pet food products (e.g., feed) provided by the device (10) are manufactured on a 1:1 basis tailored to each individual pet (40) based on gut microorganism diagnosis. This allows for the provision of optimized feed to the pet (40) without nutritional deficiencies or losses.

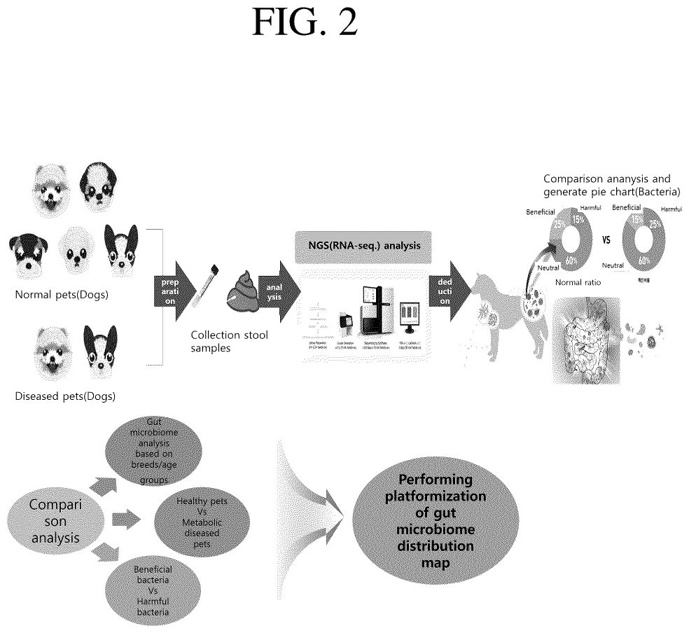

FIG. 2 is a diagram explaining the process s of constructing the community distribution map by the construction unit (13) within the device (10) for providing customized services for pets based on gut microbiome analysis according to one embodiment of the present invention.

Referring to FIG. 2, the construction unit (13) can perform microbiome analysis and derive results using stool samples from multiple pets based on comparative analysis.

To construct the community distribution map, the construction unit (13) can, for example, collect stool samples from multiple pets, perform gut microbiome analysis on the collected samples categorized by species, age group, and weight, and classify and analyze the types and community distribution of beneficial and harmful bacteria. This allows the construction unit (13) can construct the community distribution map by performing platformization of microorganisms present in the gut. Additionally, the construction unit (13) can compare and analyze the platformized microbiome map (e.g., the first map) derived from normal pets with the gut microbiome distribution (i.e., the second distribution) of pets with digestive diseases, thereby performing platformization the disease-specific microbiome map (i.e., the second map).

Based on the community distribution map, which includes the first map and the second map, the device (10) can determine the nutritional balance for each breed and individual and manufacture 1:1 customized prescription diets (customized pet food products).

For example, the construction unit (13) can collect (secure) stool samples from pets categorized by species, age group, and weight through cooperation with enterprises and current customers of its services. The construction unit (13) can perform NGS analysis on the collected stool samples to derive the distribution ratios of beneficial and harmful bacteria and construct (generate, create) the microbial distribution map, i.e., the community distribution map. The construction unit (13) can also apply AI-based deep learning data analysis (i.e., apply pre-trained AI models) to construct the community distribution map.

The AI (artificial intelligence) model used by the construction unit (13) can refer to machine learning models, neural network models (artificial neural network models), neuro-fuzzy models, etc. Additionally, various neural network models such as convolutional neural networks (CNN), recurrent neural networks (RNN), and deep neural networks (DNN), which are already known or will be developed in the future, can be applied.

Additionally, the construction unit (13) can analyze stool samples from breeds with diseases and compare the results with those from healthy breeds to create pie charts and perform platformization the results. This can be used as a foundational resource for the future manufacturing of customized functional prescription pet food.

The construction unit (13) can analyze the gut microbial community and its connection (correlation) with various diseases by analyzing stool samples from multiple pets. This includes analyzing the correlation between gut microorganisms and the pets' dietary habits and diseases, for example, deducing that “gut microorganisms can affect health status and can be changed and formed by dietary habits.”

Microorganisms that can influence the health status of pets and are changeable/formed by dietary habits may include the following, with [name-microbial phyletic line-role and characteristics]: 1) Firmicutes-one of the five major microbial phyla-diet-related microorganisms, 2) Bacteroidetes-one of the five major microbial phyla-diet-related microorganisms, 3) Proteobacteria-one of the five major microbial phyla-gut inflammation-related microorganisms, 4) Clostridium perfringens-Firmicutes phylum-gut disease-related microorganisms, 5) Blautia
Firmicutes phylum-beneficial bacteria that protect the gut, 6) Bifidobacterium, Lactobacillus-Actinobacteria phylum probiotics beneficial for gut health, 7) Enterococcus faecium-Firmicutes phylum-helps maintain gut environment but can develop antibiotic resistance if excessive.

The construction unit (13), for example, can select microorganisms that influence the gut microbial environment based on the five major representative microorganisms (Firmicutes, Bacteroidetes, Actinobacteria, Proteobacteria, Fusobacteria).

### <Firmicutes & Bacteroidetes>

Bacteroidetes abundance is higher in high-fiber diet groups compared to non-fiber diet groups, while Firmicutes are more abundant in non-fiber diet groups compared to high-fiber diet groups. The device (10) can use these ratios to create appropriate recipes for prescription diets.

At the genus level, the abundance of Bacteroidetes varies depending on high-protein, low-carbohydrate, and low-protein, high-carbohydrate diets.

Additionally, the number of Firmicutes tends to increase in high-carbohydrate, low-protein diets. Based on this, if the gut condition information of the pet (40) indicates an excessive distribution of Firmicutes, the device (10) can provide information on or manufacture and provide customized pet food products corresponding to a low-carbohydrate prescription diet.

In one embodiment, the construction unit (13) can derive results from analyzing stool samples from multiple pets, indicating that a balanced ratio of 5:5 between Firmicutes and Bacteroidetes, rather than an excessive distribution of either, is the optimal state for pets.

### <Proteobacteria>

Anaerobiospirillum, a microorganism in the Proteobacteria phylum, can cause diarrhea and bacteremia in immunocompromised individuals, but dogs carrying this microorganism are not at risk of infection. This suggests that using human standards for pet food manufacturing can lead to nutritional imbalances. Therefore, pet food should be precisely analyzed and manufactured by companies with expertise in pet nutrition.

<Clostridium perfringens>

Clostridium perfringens is associated with acute diarrhea in pets, and regular testing can help prevent and predict diarrhea through increased levels of this microorganism.

Clostridium perfringens is a microorganism that causes food poisoning in humans and requires caution, but it is commonly present in the gut of most pets and only problematic when its numbers increase significantly. Therefore, if the gut condition information of the pet (40) indicates an excessive number of Clostridium perfringens, the device (10) can manufacture and provide customized pet food products with nutrients to reduce this microorganism.

### <Blautia>

Blautia is a microorganism that prevents inflammatory diseases in the gut, protects the gut from pathogens through anti-inflammatory functions, and is involved in SCFA production, which regulates immune function. It tends to increase with a high-protein diet. If the gut condition information of the pet (40) indicates a low number of Blautia, the device (10) can provide customized recipe information or customized pet food products for a high-protein diet to increase Blautia.

### <Bifidobacterium & Lactobacillus>

Bifidobacterium and Lactobacillus are probiotics beneficial to the gut environment and are well-known beneficial bacteria essential for maintaining microbial balance. If the gut condition information of the pet (40) indicates a low number of Bifidobacterium and Lactobacillus, the device (10) can provide customized recipe information or customized pet food products, which allow high protein diet, to increase these probiotics.

<Enterococcus faecium>

Enterococcus faecium helps maintain the gut environment but can develop antibiotic resistance and reduce the efficacy of medications if it becomes overly dominant. If the gut condition information of the pet (40) indicates that the number of Enterococcus faecium is outside the acceptable range, which is preset, the device (10) can provide customized recipe information or customized pet food products, which allows high protein diet, to bring the number within the acceptable range.

Meanwhile, it has been proven that the structure and function of the microbiome in pets are similar to those in humans. This implies that the results from extensive human microbiome research can be referenced when conducting pet microbiome studies. Therefore, the device (10) can perform pet microbiome analysis by referencing previous human microbiome research results and considering the close relationship between gut microorganisms and dietary habits. Based on the microbiome analysis results of the pet (40) and the close relationship between gut microorganisms and dietary habits, the device can provide 1:1 customized functional prescription diet.

The device (10) can perform platformization (construct) the community analysis map (i.e., community distribution map) of the gut microbiome by breed, age group, and weight of pets, and use this standardized information to the normal pets for developing pet food industry, health nutrition supplements, and probiotics technology. The device (10) can also use the constructed community distribution map as a foundational resource for developing future probiotic products, enabling the manufacturing of individually customized functional prescription pet food for pets.

The microbiome (gut microbiome) considered in this invention refers to the collection and genetic information of microorganisms residing in pets and maintaining a symbiotic relationship. Depending on the living environment, the nutritional needs of pets may vary. Considering the high correlation between the pet's microbiome, health, and diseases, the device (10) can provide customized services based on the microbiome analysis results (gut condition information) and current disease information of the pet (40). By providing customized pet food products, the device (10) aims to improve the pet's constitution by allowing the pet (40) to consume these products.

The device (10) can provide a platform for customized nutritional therapy for pets, offering services to analyze and diagnose the gut condition information and current disease information of pets through a pet food platform, and provide customized services as solutions to improve the current gut condition information and current disease information. The device (10) can perform platformization health data of pets through gut microbiome diagnosis and analysis of multiple pets, and based on this, provide customized recipes, customized healthcare services, specialized medicine, supplements, functional foods, and other customized pet food products for specific pets (40).

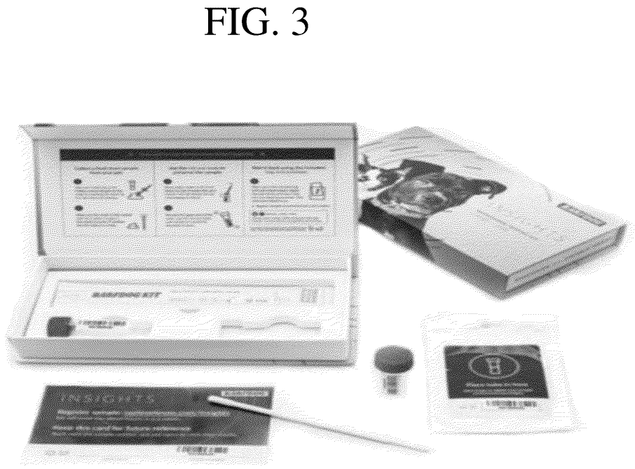

FIG. 3 is a schematic diagram showing the product shape of the diagnostic device (20) included in the system (100) for providing customized services for pets based on gut microbiome analysis according to an embodiment of the present invention.

Referring to FIG. 3, the diagnostic device (20) considered in the system (100) can be a diagnostic kit capable of diagnosing gut microorganisms, designed as a set of 4-in-1 packaged diagnostic kit, and manufactured to be recognizable as a pet product by including photos of pets. The diagnostic device (20) can be developed through research and manufactured by introducing its diagnostic equipment and systems and placing specialized personnel. This diagnostic device (20) can diagnose the presence and type of diseases (e.g., digestive diseases).

In providing customized services based on the analysis of the gut microbiome of pets (40), the device (10) can recommend and provide customized healthcare diet services through the monitoring of the pet's (40) gut microbiome (microorganisms) for individualized care management, and provide customized recipe services through microbiome analysis in stool samples. The device (10) can particularly provide optimized results in the form of GMS scores derived from various data such as gut microbial balance status, indices of beneficial and harmful bacteria, disease indices, etc., using AI models (machine learning techniques). Verified data on pets can enhance accuracy.

By providing the service of the present invention, the device (10) can build a big data repository of gut microorganisms through stool samples diagnostic analysis, secure high-quality veterinary nutritional customized healthcare data, serve as a steppingstone for entering the healthcare industry (i.e., providing customized pro/prebiotics), and enhance pet food loyalty with premium customized healthcare services.

The device (10) can also provide customized healthcare linked services, including premium health check-up and hospital linkage functions, premium customized pet food prescription functions, specialized management and treatment linkage functions for chronic diseases, obesity and diabetes management services to the pets, and premium hoteling and kindergarten management services, etc.

The service providing unit (12) can provide customized services to pets (40), such as 1:1 customized functional prescription diet. The service providing unit (12) can, for example, analyze and diagnose the ratio and distribution of beneficial and harmful bacteria in each pet's gut microbiome, identify dietary habits based on the analyzed data, and provide solutions as customized services to increase beneficial bacteria and decrease harmful bacteria. Additionally, the service-providing unit (12) can provide optimized prescription diets based on the gut microbiome analysis results for users who want customized prescription diets (e.g., customized recipes or customized pet food products) for their pets (40).

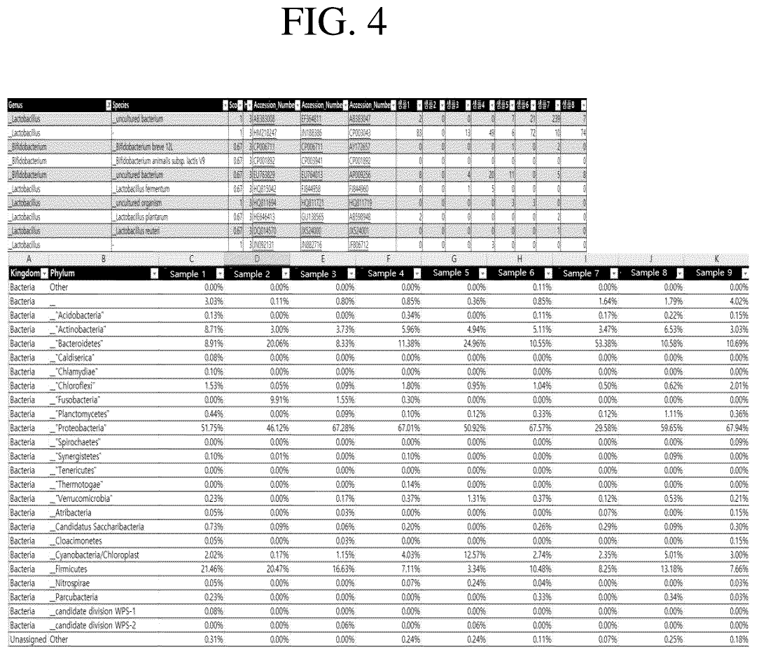

FIG. 4 is a diagram explaining the process in which the device (10) for providing customized services for pets based on gut microbiome analysis manufactures and provides probiotics as customized pet food products through gut microbiome analysis of the pet (40) according to an embodiment of the present invention.

In one embodiment, the device (10) can collect stool samples from pets (40) and perform gut microbiome analysis on the collected samples to derive gut condition information, including the types and numbers of probiotics within the pet (40). Based on the derived gut condition information, the device (10) can recommend and manufacture customized probiotics as the customized services for the user.

Referring to Table 1 of FIG. 4, it can be confirmed that the types and numbers of probiotics present in each sample (individual) from multiple pets vary. Based on the sampling data indicating different types and numbers of probiotics in each sample, the device (10) can select the necessary probiotics for each individual pet and manufacture and provide customized probiotics as customized pet food products for the pet (40).

Referring to Table 2 of FIG. 4, when gut microbiome analysis is performed on stool samples from multiple pets, information such as the types and numbers of various microorganisms present in each pet's gut (i.e., information about the types and numbers of microorganisms in each individual) can be confirmed. Based on this information, the device (10) can identify each individual's weaknesses and recommend suitable dietary habits for the pet (40) as at least one customized service. The device can also manufacture and provide customized prescription diets (tailored pet food products) as another customized service.

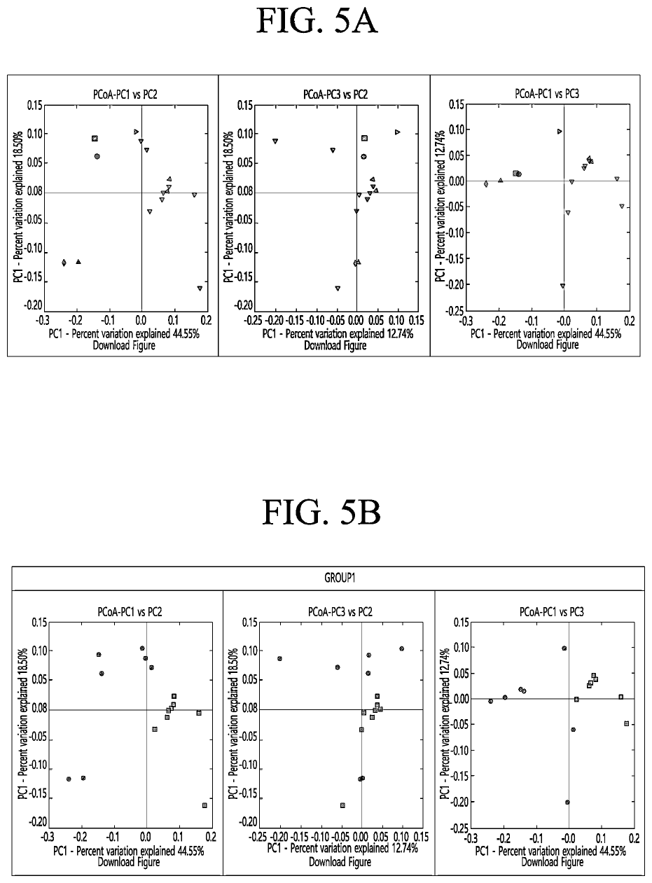

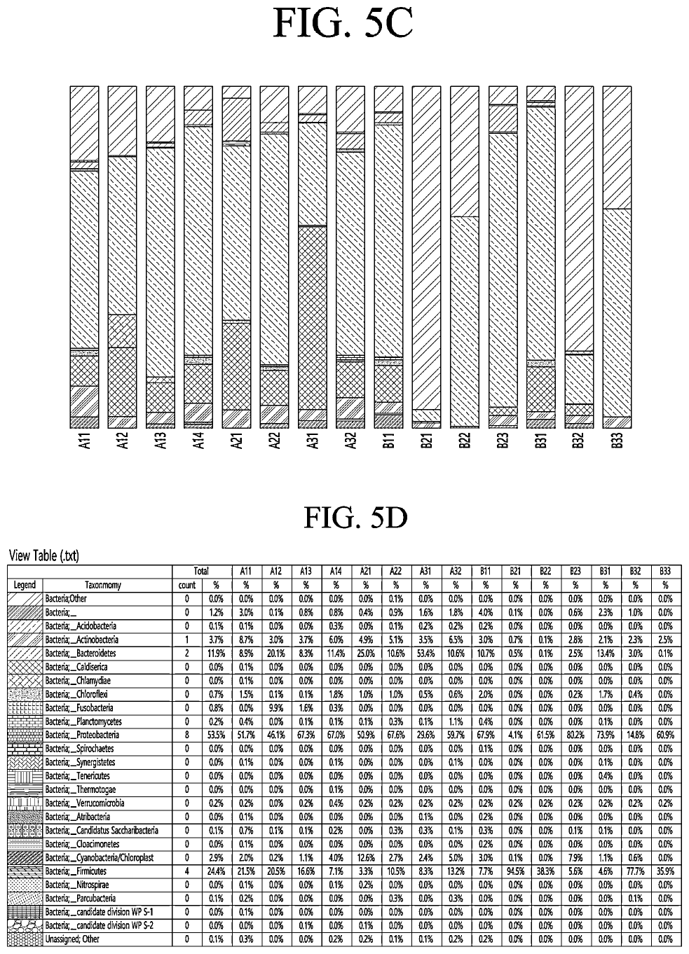

FIGS. 5A-5D are diagrams showing examples of visualized data for each sample by the device (10) for providing customized services for pets based on gut microbiome analysis according to one embodiment of the present invention.

Referring to FIGS. 5A-5D, the construction unit within the device, for example, can perform various visualizations for each sample (i.e., stool samples from multiple pets) as shown in FIGS. 5A-5D, enabling the generation and platformization of microbial community maps and distribution maps, and bio maps through analysis.

Additionally, the construction unit (13) can perform platfomization the information system of beneficial bacteria community distribution by analyzing gut microbiome data by species and age of pets and further analyze the correlation with diseases based on the constructed information, enabling the manufacturing of individually customized functional pet food and providing enhanced healthcare services.

Moreover, in the process of constructing the community distribution map, the construction unit (13) can prepare and analyze samples from at least two types of metabolic diseases (constipation, diarrhea, etc.) and normal groups, and perform gut beneficial bacteria community distribution analysis using next-generation sequencing (NGS). The construction unit (13) can then derive results of next-generation sequencing (NGS) analysis from the prepared various samples and identify beneficial bacteria (Lactobacillus, Firmicutes, Enterococcus faecium, Clostridium, Proteobacteria, etc.) the from results. Furthermore, the construction unit (13) can conduct gut microbiome analysis in various breed groups through sample provision by companies and provide platformized community distribution information map data of microorganisms (i.e., community distribution map) to companies. The construction unit (13) can also perform precise analysis using AI models if necessary.

Additionally, in one embodiment, the construction unit (13) of the device (10) can analyze the correlation between saliva and stool samples from multiple pets. The construction unit (13) can analyze the correlation between disease and inflammation-related biomarkers in stool and inflammation-related biomarkers in saliva through microbiome research using stool samples and derive information on the correlation between oral and gut microbiome and diseases. Based on this, the construction unit can provide the customized services. The construction unit (13) can also collect disease history through stool sampling (stool samples) and, simultaneously, analyze the correlation between microbiome in stool and gut and inflammation-related biomarkers, etc., from all microorganisms in sampled saliva and oral.

The device (10) can develop and provide customized diets based on gut microbial community data for each pet disease, offer scientifically based customized solutions for diseases, increase the revenue of related companies through the manufacturing of functional nutritional pet food, reduce medical costs through improved pet healthcare, and increase employment in the related industry through revitalization of pet industry. Additionally, the device (10) can improve the mental quality of life of pet owners by promoting pet health and extending the lifespan of pets through the construction of gut microbial ecosystem maps (i.e., community distribution maps).

Additionally, the device (10) can provide customized diets based on subjective survey responses (medical examination by interview) predicted by users, who are the pet (40) owners, received from user terminals (30). The device (10) can provide 1:1 customized functional prescription diet to pets (40) based on actual data obtained through the diagnostic device (20), diagnose the current health status of the pets, recommend diets accordingly, and suggest potential treatment and improvement options. It can also offer comprehensive supplements, health, nutrition, and premium services tailored to life stages.

Moreover, the subscription management unit (16) within the device (10) can offer a subscription delivery service where premium fresh ingredients (organic, eco-friendly, etc.) are used to custom-make fresh food (raw food) for pets, delivered through an online subscription service. By providing the subscription delivery service, the device (10) can allow the users to conveniently compose pet food ingredients and add supplements through an online service, with options to choose the time and place for delivery by themselves.

The device (10) can also offer a web app platform in beta service as an online pet food platform where users can access professional and useful information on pet health, nutrition, and life stages and purchase customized fresh pet food. It can manufacture customized pet food products, create a community (blog) to continually reflect customer needs, and establish hygienic manufacturing facilities.

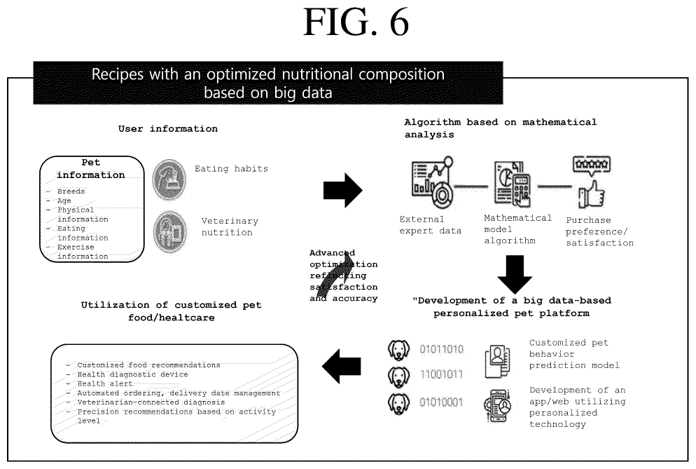

FIG. 6 is a diagram explaining the customized services provided by the device (10) for providing customized services for pets based on gut microbiome analysis according to an embodiment of the present invention.

Referring to FIG. 6, the device (10) can receive pet information such as breed, age, physical information (weight, etc.), dietary habits), information (eating and exercise information (activity level) from user terminals (30). Users can also input information related to dietary habits such as bowel movements and diet status, and veterinary nutritional information such as disease presence and nutritional composition through the device (10). The device (10) can analyze user preferences and satisfaction with purchases through a numerical analysis-based algorithm and develop a pet-customized platform based on big data, including predicting pet preferences and developing a customized app/web platform. The device (10) can provide the customized services such as personalized food recommendations, provision of tailored health diagnostic devices, provision of health alerts, automatic ordering and delivery date management, function of veterinary diagnosis connection, and detailed analysis recommendations based on activity levels, based on the acquired pet information and big data analysis.

That is, when the device (10) receives pet information from user terminals (30), it can manufacture customized fresh food (customized pet food products) based on the received pet information and deliver it regularly to the user. Additionally, the device can provide customized diets and care services tailored to the pet (40).

The device (10) can manufacture and provide functional, and prescription customized food based on individual microbiome analysis of pets. It can provide services that offer customized diets to pets through surveys conducted with pet owners and can also provide precise customized diets to pets (40) through the diagnostic device (20). Based on the various information stored in the database unit (15), the device (10) can evaluate the pet's condition before and after subscribing to a specific customized pet food product and recommend different diets to the user according to the evaluation results. This way, the device (10) can provide more accurate and evidence-based diets to the pet (40), ensuring that the service is trustworthy and reliable for the user (customer).

Additionally, in one embodiment, the device (10) can provide the following functions. The control unit (17) can offer an animal hospital reservation service, allowing users to book and visit offline animal hospitals.

Specifically, the control unit (17) detects that the user has selected the veterinary hospital reservation button provided in an area of the screen of the user terminal (30) for providing the veterinary hospital reservation service. Upon detection, it can display veterinary hospital map information, which shows the locations of multiple veterinary hospitals existing offline, on the screen of the user terminal (30). Subsequently, the control unit (17) allows the user to select the icon of a specific veterinary hospital (the first veterinary hospital) among the multiple veterinary hospitals displayed on the veterinary hospital map information, for which the user wishes to make a visit. It then enables the user to create an appointment request data for a medical consultation. The appointment request data created by the user can then be stored and managed in the reservation database (not shown) within this device (10).

The appointment request data can include, for example, the visitor's name, the desired visit date and time, information about the first animal hospital (such as the hospital name, address, phone number) where the user wishes to visit, and data about the pet (40), such as breed, age, and weight.

After the appointment request data created by the user is stored in the reservation database (not shown), the control unit (17) can monitor the stored appointment request data. For example, when the day corresponding to the appointment date arrives, it can provide an appointment notification message on the screen of the user terminal (30). For instance, the appointment notification message might be information such as, ‘Today is the appointment day at the first veterinary hospital. Please visit on time,” etc.

Additionally, when the day arrives, the control unit (17) can provide an appointment information notification to the veterinary hospital terminal held by the staff of the corresponding veterinary hospital (i.e., the first veterinary hospital) based on the information in the appointment request data. Here, the appointment information notification might be something like, “User A has an appointment at 2 PM.” To achieve this, the device (10) can be connected to the veterinary hospital terminal (not shown) via a network to send and receive data, and it can control the operation of both the veterinary hospital terminal and the user terminal.

At this time, in the past, some users would make reservations at veterinary hospitals but often fail to show up for the appointment, a phenomenon known as “no-show.” Such no-shows cause financial and time-related damages to the veterinary hospitals (or the reservation-associated businesses).

To prevent such as no-shows, the control unit (17) can, for example, perform a determination procedure to assess the possibility of a no-show by the user when the day corresponding to the appointment date in the appointment request data arrives.

Specifically, to perform the determination procedure, when the day corresponding to the appointment date in the appointment request data arrives, the control unit (17) can detect whether the GPS sensor of the user terminal (30) is OFF at a predetermined time (e.g., 30 minutes) before the appointment time (e.g., 2:00 PM), which is the determination time (e.g., 1:30 PM). If the GPS sensor is detected to be OFF at the determination time, the control unit (17) forcibly turns it ON to obtain real-time location information (i.e., GPS information) of the user terminal (30) for a predefined measurement period (e.g., 2 minutes) from the determination time. Afterward, the control unit forcibly turns the GPS sensor OFF again. Based on the real-time location information obtained, it can generate movement trajectory information of the user terminal (30) corresponding to the measurement period.

At this time, the user may be a registered member of the service provided by this device (10). When the user registers with this device (10), the control unit (17) can obtain prior consent from the user for the forced collection of location information from the user terminal (30) by forcibly controlling the ON/OFF state of the GPS sensor on the user terminal (30), and for the use of the collected location information. This consent data can be stored in the database unit (15) of this device (10). Based on this data, the control unit (17) can forcibly turn the GPS sensor of the user terminal (30) ON at around the determination time or turn it OFF afterward. For example, this device (10) can allow only those users who have provided the aforementioned consent data to use specific services, such as the veterinary hospital reservation service, among the services offered.

Meanwhile, after generating the movement trajectory information, the control unit (17) can determine if the movement trajectory information (including entire movement trajectory information) is located within a preset distance range (e.g., within 1.5 km) from the location of the first veterinary hospital on the map based on the veterinary hospital map information. If it is determined that the entire movement trajectory information is within this range, it can be judged that the user has a low or no possibility of a no-show. Subsequently, the control unit can generate first determination result information indicating the low possibility of a no-show and transmit it to the veterinary hospital terminal (not shown). For example, the first determination result information might be in a message form like, “User A, who has an appointment at 2 PM, is currently within a 1.5 km radius, indicating a low possibility of a no-show. The user is likely to arrive soon,” etc.

On the other hand, if the control unit (17) determines that the entire movement trajectory information is not within the preset distance range (i.e., if at least part of the movement trajectory information is outside the preset distance range), it can derive the first direction-related information (i.e., the direction in which the user terminal is moving according to the movement trajectory information) based on the veterinary hospital map information. Subsequently, the control unit (17) can derive the second direction-related information, which is the direction from the starting position of the location information within the movement trajectory information towards the location of the first veterinary hospital on the veterinary hospital map. Then, the control unit perform (17) can a direction consistency determination process to judge whether the two derived direction-related information (i.e., the first and second direction-related information) correspond to each other (i.e., whether they are in the same direction).

When the control unit (17) determines the direction consistency process, as a result of the direction consistency determination process, that the two direction-related information correspond to each other (e.g., both direction-related information indicate a movement from east to west), it can derive the estimated travel time from the last position of the location information within the movement trajectory information to the veterinary hospital location based on the veterinary hospital map information. In this case, by analyzing the position change information within the movement trajectory information, if it is determined that the user is walking, the control unit (17) can derive the estimated travel time based on walking. If it is determined that the user is using a means of transportation such as a taxi, the control unit (17) can derive the estimated travel time based on the transportation method. Here, the estimated travel time refers to the information predicting the time required for the travel.

When the control unit (17) finds that both the estimated travel time based on walking and the estimated travel time based on the transportation method are, for example, 25 minutes and 13 minutes respectively, and both are shorter than a preset time considered at the determination time (i.e., 30 minutes), it can determine that, although the user who has made an appointment at the first veterinary hospital is currently outside the preset distance range (i.e., the user is somewhat far from the veterinary hospital), the user can still arrive at the first veterinary hospital by the appointment time (e.g., 2:00 PM). Subsequently, the control unit (17) can generate the first determination result information indicating a low possibility of a no-show and transmit it to the veterinary hospital terminal, as previously described.

However, if the control unit (17) determines that i) the two direction-related information correspond to each other, but both the estimated travel times are, for example, 2 hours and 50 minutes respectively (i.e., the walking-based estimated travel time is 2 hours and the transportation-based estimated travel time is 50 minutes), which are both longer than the preset time (i.e., 30 minutes) considered at the determination time, or ii) the two direction-related information do not correspond to each other, and both the estimated travel times are longer than the preset time (i.e., 30 minutes), it can determine that the user who made an appointment at the first veterinary hospital is currently outside the preset distance range and far from the veterinary hospital, which means that even if the user immediately starts moving towards the veterinary hospital using a means of transportation such as a taxi, the user cannot arrive at the first veterinary hospital by the appointment time (e.g., 2:00 PM), subsequently, the control unit can generate second determination result information indicating a high possibility of the no-show and transmit it to the veterinary hospital terminal.

Here, the second determination result information might be in a message form like, “User A, who has an appointment at 2:00 PM, is currently located far away, making it unlikely that they will visit before 2:00 PM. Therefore, the likelihood of a no-show is high. Verification is needed.” Upon receiving the second determination result information, the hospital staff with the veterinary hospital terminal can, for example, call the user terminal (30) based on the received second determination result information to confirm whether the user can visit by the scheduled appointment time.

Additionally, the control unit (17) can simultaneously generate confirmation guidance information to check the visit possibility to the user terminal (30) upon generating and transmitting the second determination result information to the veterinary hospital terminal. The confirmation guidance information can be displayed on the screen of the user terminal (30). Depending on the type of response from the user to the confirmation guidance information, the control unit can further generate third determination result information and transmit it to the veterinary hospital terminal.

At this time, after providing the confirmation guidance information on the screen, the control unit (17) can generate and transmit third determination result information to the veterinary hospital terminal based on the user's response to the confirmation guidance information. Depending on the situation, the third determination result information transmitted to the veterinary hospital terminal based on the user's response can be one or more of the following three:
i) If the user responds to the confirmation guidance information with “Possible,” the control unit can generate third determination result information, including information such as “The user indicated they can visit at the scheduled time,” and transmit it to the veterinary hospital terminal.
ii) If the user responds to the confirmation guidance information with “Not possible (rescheduling needed),” the control unit can generate third determination result information, including information such as “The user indicated they cannot visit at the scheduled time and need to reschedule,” and transmit it to the veterinary hospital terminal.
iii) If the user does not respond to the confirmation guidance information (i.e., “No response”), the control unit can third determination result information, including generate information such as “The user did not respond, indicating a very high possibility of a no-show,” and transmit it to the veterinary hospital terminal.

After transmitting the third determination result information to the veterinary hospital terminal, the control unit (17) can complete the execution of the described determination procedure.

This device (10) can provide a veterinary hospital reservation service to users and, through the aforementioned determination procedure, assess the likelihood of a no-show by the user. The assessed information can be provided to the veterinary hospital staff, allowing the user to make appointments at veterinary hospitals more conveniently. Additionally, it offers convenience in performing and managing appointments for both the hospital staff and the user.

Below, we will briefly examine the operation flow of the present invention based on the detailed description provided above.

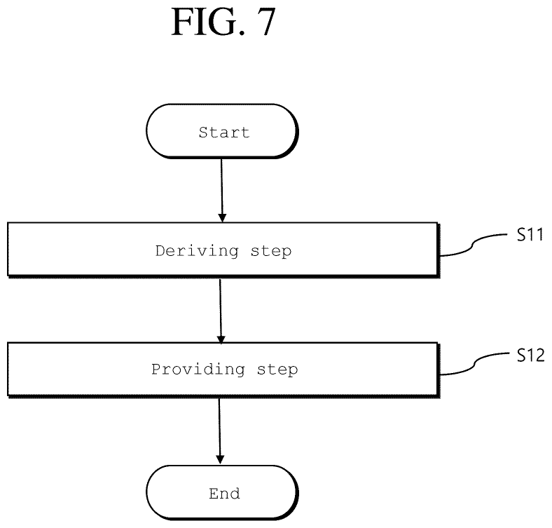

FIG. 7 is a flowchart illustrating a method for providing a customized pet service based on gut microbiome analysis according to an embodiment of the present invention.

The method for providing a customized pet service based on gut microbiome analysis shown in FIG. 7 can be performed by the aforementioned device (10). Therefore, even if certain details are omitted below, the content described regarding the device (10) can be equally applied to the explanation of the method for providing a customized pet service based on gut microbiome analysis.

Referring to FIG. 7, in step S11, the analysis unit can derive the gut condition information of the pet through gut microbiome analysis targeting the pet.

At this time, the analysis unit can use a pre-manufactured portable diagnostic device to collect the stool samples from the pet. After performing gut microbiome analysis on the collected fecal sample, the analysis unit can derive analysis result information on the microorganisms, including beneficial and harmful bacteria in the pet's gut, as the gut condition information.

Next, in step S12, the service providing unit can provide a customized service tailored to the pet based on the gut condition information.

Additionally, the service providing unit can provide the customized recipe information that matches the gut condition information, or information about customized pet food products manufactured based on the customized recipe information, as part of the customized service.

In the aforementioned description, steps S11 and S12 can be further divided into additional steps or combined into fewer steps according to embodiments of the present invention. Additionally, some steps may be omitted if necessary, and the order of the steps can be changed.

In the one embodiment, the method for providing a customized pet service based on gut microbiome analysis of the present invention can be implemented in the form of program instructions executable through various computer means and recorded on a computer-readable medium. The computer-readable medium can include program instructions, data files, and data structures either individually or in combination. The program instructions recorded on the medium can be those specially designed and configured for the present invention or those known and available to those skilled in the art. Examples of computer-readable recording media include magnetic media such as hard disks, floppy disks, and magnetic tapes, optical media such as CD-ROMs and DVDs, magneto-optical media such as floptical disks, and hardware devices specially configured to store and execute program instructions such as ROM, RAM, and flash memory. Examples of program instructions include machine code such as that generated by a compiler, as well as high-level language code executable by a computer using an interpreter, etc. The aforementioned hardware devices can be configured to operate as one or more software modules to perform the operations of the present invention, and vice versa.

Additionally, the aforementioned method for providing a customized pet service based on gut microbiome analysis can also be implemented in the form of a computer program or application executed by a computer and stored on a recording medium.

The above description of the present invention is intended as an example, and those skilled in the art will understand that various modifications can be made in other specific forms without altering the technical spirit or essential characteristics of the present invention. Therefore, the embodiments described above should be understood as illustrative in all aspects and not restrictive. For example, each component described as a single unit may be implemented in a distributed manner, and similarly, components described as distributed may be implemented in a combined form.

The scope of the present invention is indicated by the claims below rather than the foregoing detailed description, and all changes or modifications derived from the meaning and scope of the claims and their equivalents should be interpreted as being included within the scope of the present invention.

What is claimed is:

1. A device for providing customized services for pets based on gut microbiome analysis, comprising: an analysis unit to derive information about the gut condition of the pet through gut microbiome analysis; and a service providing unit to offer customized services for the pet based on the gut condition information.

2. The device of claim 1,
wherein the analysis unit to collect a stool sample from the pet using a pre-manufactured diagnostic device, performs gut microbiome analysis on the collected stool sample, and to analyze result information about microorganisms, including beneficial and harmful bacteria in the pet's gut, as the gut condition information.

3. The device of claim 1,
further comprising a construction unit for establishing a microbiome-based community distribution map through the analysis of stool samples from multiple pets,
wherein the analysis unit to derive gut condition information using the established community distribution map.

4. The device of claim 1,
wherein the service providing unit to provide customized recipe information according to the gut condition information or customized pet food product information manufactured based on the customized recipe information, as the customized service.

5. The device of claim 1,
further comprising an acquisition unit to obtain pet information from a user terminal, the pet information including at least one of the breeds, age, weight, eating habits, and activity level of the pet, and
wherein the service providing unit to provide the customized service by further considering the pet information.

6. The device of claim 1,
further comprising a subscription management unit to regularly deliver the customized pet food products on pre-assigned delivery dates when a subscription request for information about customized pet food products is made from the user terminal.

7. A method for providing customized services for pets based on gut microbiome analysis using the device of claim 1, comprising: deriving gut condition information of the pet through gut microbiome analysis in the analysis unit; and providing a customized service for the pet based on the gut condition information in the service providing unit.
